(19) United States
(12) Patent Application Publication
(10) Pub. No.: US 2021/0100621 A1
(43) Pub. Date: Apr. 8, 2021

(54) Surgical Navigation System For Registering Coordinates of Patient-Customized Tool

(71) Applicants: Kyungpook National University Industry-Academic Cooperation Foundation, Daegu (KR); Korea Advanced Institute of Science and Technology, Daejeon (KR)

(72) Inventors: Jun Young KIM, Daegu (KR); Shin Yoon KIM, Daegu (KR); Anna SEO, Daegu (KR); Hyun Mun KIM, Seoul (KR); Hyun Deok KIM, Daegu (KR); Min Kyu JE, Daegu (KR); Jae Suk CHOI, Gyeonggi-do (KR)

(21) Appl. No.: 15/733,200

(22) PCT Filed: Dec. 12, 2018
(86) PCT No.: PCT/KR2018/015482

§ 371 Date: Jun. 9, 2020

(30) Foreign Application Priority Data
Dec. 12, 2017 (KR) 10-2017-0170779

(51) Int. Cl. A61B 34/20; A61B 5/06

(52) U.S. Cl. CPC A61B 34/20; A61B 2034/2051; A61B 5/062

(57) ABSTRACT

A surgical navigation system for registering coordinates of a patient-customized tool, according to one embodiment, comprises: an electromagnetic wave generation unit for generating an electromagnetic wave; an electromagnetic sensor provided in a patient-customized tool and including a coil through which the electromagnetic wave passes; and a processing unit for calculating the position and the orientation of the electromagnetic sensor on the basis of magnetic flux, which is interlinked with the coil, and calculating the position and the orientation of the patient-customized tool on the basis of the position and the orientation of the electromagnetic sensor.

The following example embodiments relate to a surgical navigation system for registering coordinates of a patient-customized tool (hereinafter, referred to as a “patient-specific instrument (PSI)”).

### BACKGROUND ART

In surgery of many fields, including an orthopedic surgical operation, clinicians require a surgical navigation system that visualizes an invisible internal part of a human body. The surgical navigation system uses a scheme of tracking a position of a surgical instrument, and the like, by matching coordinates of an actual space where a human body is located and coordinates of a three-dimensional (3D) virtual space generated by a computer aided system. Such a scheme requires an absolute position registration of a surgical instrument, and the like.

For the absolute position registration of the surgical instrument, and the like, various conventional methods have been proposed. As an example, there is a method of attaching optical sensors to a surgical instrument and an affected part of a human body, of detecting light reflected from the optical sensors using a camera in response to light being irradiated to each of the optical sensors, and of obtaining a relative position relationship between the surgical instrument and the affected part of the human body using a computer. In the above method, a size of the optical sensor needs to be greater than that of other types of sensors (e.g., an electromagnetic sensor), and a path of light needs to be secured so that light irradiated to the optical sensor is not blocked by an obstacle. For example, Korean Patent Application Publication No. 10-2016-0042297 discloses a medical navigation device.

### DISCLOSURE OF INVENTION

### Technical Goals

An aspect provides a surgical navigation system for registering coordinates of a patient-specific instrument (PSI) by installing an electromagnetic sensor for sensing an electromagnetic wave in the PSI and by calculating position information of the PSI based on information sensed by the electromagnetic sensor, to register the coordinates of the PSI.

### Technical Solutions

According to an aspect, there is provided a surgical navigation system for registering coordinates of a patient-specific instrument (PSI) including: an electromagnetic wave generation unit configured to generate an electromagnetic wave; an electromagnetic sensor installed in the PSI, the electromagnetic sensor including a coil through which the electromagnetic wave passes; and a processing unit configured to calculate a position and an orientation of the electromagnetic sensor based on magnetic flux interlinked with the coil, and to calculate the position and the orientation of the PSI based on the position and the orientation of the electromagnetic sensor.

The processing unit may be configured to calculate the position and the orientation of the electromagnetic sensor based on frequencies orthogonal to each other in the magnetic flux interlinked with the coil.

The processing unit may be configured to calculate the magnetic flux based on an induced electromotive force generated in the coil in response to the electromagnetic wave passing through the coil.

The processing unit may be integrated with the electromagnetic sensor to be installed together in the PSI.

The electromagnetic sensor may further include an induced electromotive force detection unit configured to detect an induced electromotive force generated in the coil; and an analog-to-digital conversion unit configured to convert the induced electromotive force from an analog form to a digital form.

According to an aspect, there is provided a surgical navigation system for registering coordinates of a PSI including: an electromagnetic wave generation unit configured to generate an electromagnetic wave; a plurality of electromagnetic sensors installed in the PSI, each of the plurality of electromagnetic sensors including a coil through which the electromagnetic wave passes, and coils being installed in different positions and orientations in different planes of the PSI; and a processing unit configured to calculate a position and an orientation of each of the plurality of electromagnetic sensors based on magnetic flux interlinked with each of the coils and to calculate the position and the orientation of the PSI based on the position and the orientation of each of the plurality of electromagnetic sensors.

The processing unit may be configured to calculate the position and the orientation of each of the plurality of electromagnetic sensors based on frequencies orthogonal to each other in the magnetic flux interlinked with each of the coils.

The processing unit may be configured to calculate the magnetic flux interlinked with each of the coils based on each induced electromotive force generated in each of the coils in response to the electromagnetic wave passing through each of the coils.

The plurality of electromagnetic sensors may be spaced apart from each other at a set angular interval with respect to the PSI and may be installed in different planes.

### Effects

According to example embodiments, a surgical navigation system for registering coordinates of a patient-specific instrument (PSI) may register coordinates of a PSI by installing an electromagnetic sensor for sensing an electromagnetic wave in the PSI, without a limitation of a movement of a clinician, unlike a conventional optical sensor that necessarily requires securing of a line of sight.

According to example embodiments, even though a PSI has the uneven surface structure a surgical navigation system for registering coordinates of a PSI may accurately calculate the position and the orientation of a PSI by installing a plurality of electromagnetic sensors in the uneven surface structure.

According to example embodiments, in a surgical navigation system for registering coordinates of a PSI, an electromagnetic sensor may be integrated with a processing unit configured to process a signal sensed by the electromagnetic sensor, and the electromagnetic sensor and the processing unit may be installed together in a PSI, to miniaturize a volume of a sensor and simplify use of a conventional surgical navigation system.

The effects of the surgical navigation system are not limited to the above-mentioned effects. Also, other unmentioned effects can be clearly understood from the following description by those having ordinary skill in the technical field to which the present disclosure pertains.

### BRIEF DESCRIPTION OF DRAWINGS

FIG. 1 is a block diagram schematically illustrating a surgical navigation system for registering coordinates of a patient-specific instrument (PSI) according to an example embodiment.

FIG. 2 is a block diagram schematically illustrating a surgical navigation system for registering coordinates of a PSI according to an example embodiment.

FIG. 3 is a perspective view schematically illustrating a surgical navigation system for registering coordinates of a PSI according to an example embodiment.

FIG. 4 is a flowchart schematically illustrating a method of registering coordinates of a PSI in a surgical navigation system for registering coordinates of a PSI according to an example embodiment.

### BEST MODE FOR CARRYING OUT THE INVENTION

Hereinafter, example embodiments will be described in detail with reference to the illustrative drawings. Regarding the reference numerals assigned to the components in the drawings, it should be noted that the same components will be designated by the same reference numerals, wherever possible, even though they are shown in different drawings. Further, in the following description of the example embodiments, a detailed description of publicly known configurations or functions incorporated herein will be omitted when it is determined that the detailed description obscures the subject matters of the example embodiments.

In addition, the terms first, second, A, B, (a), and (b) may be used to describe constituent elements of the example embodiments. These terms are used only for the purpose of discriminating one constituent element from another constituent element, and the nature, the sequences, or the orders of the constituent elements are not limited by the terms. When one constituent element is described as being “connected”, “coupled”, or “attached” to another constituent element, it should be understood that one constituent element can be connected or attached directly to another constituent element, and an intervening constituent element can also be “connected”, “coupled”, or “attached” to the constituent elements.

The constituent element, which has the same common function as the constituent element included in any one example embodiment, will be described by using the same name in other example embodiments. Unless disclosed to the contrary, the configuration disclosed in any one example embodiment may be applied to other example embodiments, and the specific description of the repeated configuration will be omitted.

The term “patient-specific instrument (PSI)” used herein refers to an instrument inserted into an affected part (e.g., acetabulum) of a human body in a surgical operation (e.g., a total joint replacement), and is used to register an absolute position of the affected part of the human body to set an axis of another surgical instrument (e.g., a reamer) to be inserted into an affected part of a patient.

The term “orthogonal elements” used herein refers to principal elements (e.g., (x, y, z), and (r, θ, φ) of a specific coordinate system (e.g., a Cartesian coordinate system, a spherical coordinate system, and the like), which have a physical quantity (e.g., an electromagnetic field, magnetic flux, and the like) expressed by the orientational vector.

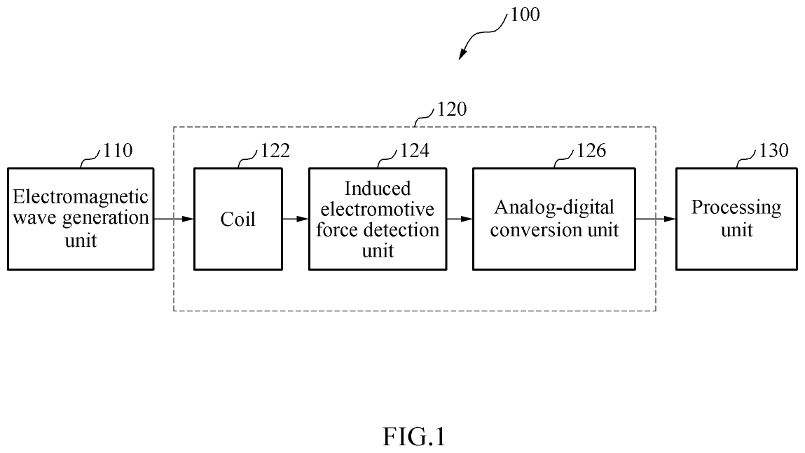

FIG. 1 is a block diagram schematically illustrating a surgical navigation system for registering coordinates of a PSI according to an example embodiment.

Referring to FIG. 1, a surgical navigation system 100 for registering coordinates of a PSI according to an example embodiment may sense, using an electromagnetic sensor 120 installed in a patient-specific instrument, an electromagnetic wave generated from an electromagnetic wave generation unit 110, and may calculate the position and the orientation of a PSI through a series of signal processing, to register absolute coordinates of the PSI. Here, the absolute coordinates of the PSI may be represented by principal components of a coordinate system of a space in which the surgical navigation system 100 is placed.

The surgical navigation system 100 may include the electromagnetic wave generation unit 110, the electromagnetic sensor 120, and a processing unit 130 that processes a signal.

The electromagnetic wave generation unit 110 may generate an electromagnetic field. In the present disclosure, the electromagnetic field may be referred to as an electromagnetic wave. The electromagnetic wave generation unit 110 may be disposed outside the PSI, unlike the electromagnetic sensor 120.

The electromagnetic sensor 120 may sense an electromagnetic field generated from the electromagnetic wave generation unit 110, and may generate information about the position and the orientation of the electromagnetic sensor 120 based on the sensed electromagnetic field, or a signal required to generate the information. The electromagnetic sensor 120 may include a coil 122 through which the electromagnetic field passes, an induced electromotive force detection unit 124, and an analog-to-digital conversion unit 126.

The induced electromotive force detection unit 124 may detect an induced electromotive force generated in the coil 122 when the electromagnetic wave generated in the electromagnetic wave generation unit 110 passes through the coil 122. For example, the induced electromotive force detection unit 124 may be a voltage sensor connected to both ends of the coil 122. In an example embodiment, the induced electromotive force detection unit 124 may detect an induced electromotive force corresponding to a principal frequency. Since the electromagnetic field is a physical quantity with directivity, the electromagnetic field may be represented by principal elements of a set coordinate system. For example, in a Cartesian coordinate system, an electromagnetic field B may be represented as (B1, B2, B3). Here, elements B1, B2 and B3 of the electromagnetic field may be orthogonal to each other. When the electromagnetic field B passes through the coil 122, the induced electromotive force detection unit 124 may detect an induced electromotive force of a principal frequency (e.g., frequencies f1, f2 and f3) corresponding to each of the elements B1, B2 and B3 of the electromagnetic field.

The analog-digital conversion unit 126 may convert an analog induced electromotive force detected by the induced electromotive force detection unit 124 into a digital signal. When the digital signal is used instead of the analog induced electromotive force, an energy efficiency greater than an energy efficiency obtained when processing an analog signal may be achieved.

The processing unit 130 may calculate the position and the orientation of the electromagnetic sensor 120 based on magnetic flux interlinked with the coil 122, according to the induced electromotive force detected by the induced electromotive force detection unit 124. The processing unit 130 may calculate the position and the orientation of the PSI based on the calculated position and the calculated orientation of the electromagnetic sensor 120. Information about the calculated position and the calculated orientation of the PSI is used to register coordinates of the PSI.

In an example embodiment, in response to the induced electromotive force detection unit 124 detecting an induced electromotive force corresponding to a principal frequency, the magnetic flux interlinked with the coil 122 may also have directivity corresponding to the principal frequency. The processing unit 130 may calculate the position and the orientation of the electromagnetic sensor 120 based on principal frequencies of the magnetic flux interlinked with the coil 122, that is, orthogonal frequencies, for example, a first frequency f1, a second frequency f2 and a third frequency f3. Since magnetic flux of the first frequency f1, magnetic flux of the second frequency f2 and magnetic flux of the third frequency f3 have orientation orthogonal to each other, the position and the orientation of the electromagnetic sensor 120 with respect to the electromagnetic wave generation unit 110 may be calculated based on the magnetic fluxes.

In an example embodiment, the processing unit 130 may calculate magnetic flux based on an induced electromotive force generated in the coil 122 when the electromagnetic field passes through the coil 122. For example, information about a number of turns of the coil 122 may be used.

In an example embodiment, the electromagnetic sensor 120 is installed in the PSI. The PSI is inserted into an affected part of a pre-operative patient, is used to register coordinates of a position of the affected part in a coordinate system in a surgical navigation system, and may be removed during an intra-operative period. In other words, installing of the electromagnetic sensor 120 in the PSI indicates that a correct insertion path of an instrument used during surgery based on registration of the coordinates of the position of the affected part of the pre-operative patient may be guided, and that a possibility of distortion of a signal of surgical equipment that is an electronic device during the surgery may be fundamentally blocked.

In an example embodiment, although not shown, the electromagnetic sensor 120 may include the processing unit 130. In other words, the processing unit 130 integrated with the electromagnetic sensor 120 may be embedded in the electromagnetic sensor 120. In this example, the electromagnetic sensor 120 and the processing unit 130 may be installed together in the PSI. In this example, the electromagnetic sensor 120 may further include a wireless communication unit (not shown). The wireless communication unit may wirelessly transmit information about the position and the orientation of the PSI calculated by the processing unit 130 to an external data receiver unit. For example, the wireless communication unit may use wireless fidelity (WiFi), Bluetooth, near field communication (NFC). Although not shown, an external processing unit (e.g., a computer) may receive the information about the position and the orientation of the PSI and may register coordinates of the position of the PSI.

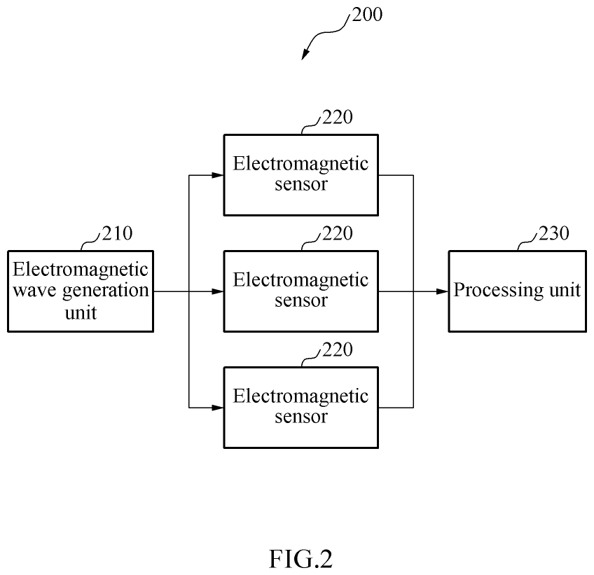

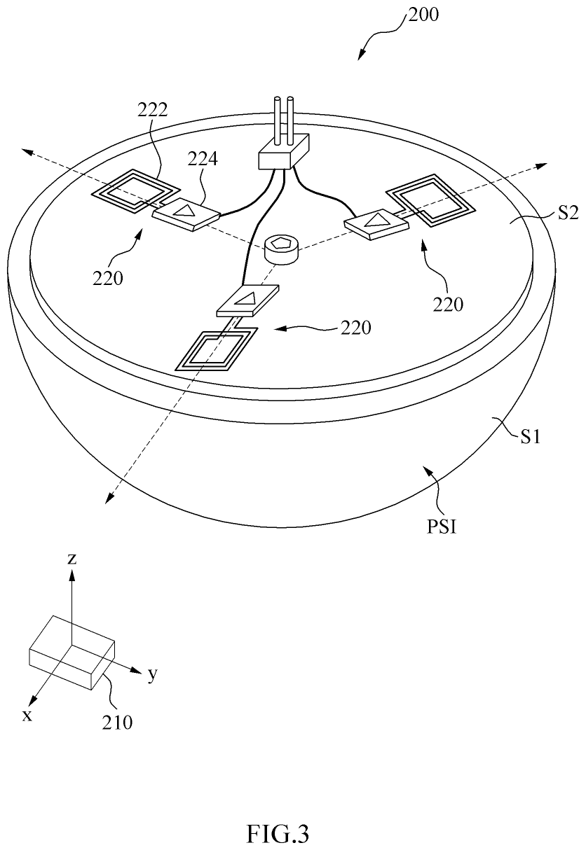

FIG. 2 is a block diagram schematically illustrating a surgical navigation system for registering coordinates of a PSI according to an example embodiment, and FIG. 3 is a perspective view schematically illustrating a surgical navigation system for registering coordinates of a PSI according to an example embodiment.

Referring to FIGS. 2 and 3, a surgical navigation system 200 for registering coordinates of a PSI according to an example embodiment may calculate the position and the orientation of a PSI by installing a plurality of electromagnetic sensors 220 in an uneven surface structure of the PSI based on the uneven surface structure. The surgical navigation system 200 may include an electromagnetic wave generation unit 210, the plurality of electromagnetic sensors 220, and a processing unit 230. As described above, a plurality of processing units 230 may be embedded in the plurality of electromagnetic sensors 220, respectively.

The PSI may have a first surface S1 inserted into an affected part of a patient, and a second surface S2 disposed on an opposite side of the first surface S1. For example, the first surface S1 of the PSI may have a shape corresponding to a shape of an acetabulum. Since the PSI has a shape corresponding to a shape of an affected part of a patient as described above, the PSI may have an uneven surface structure. In the following description, for convenience of description, the second surface S2 is an uneven surface, and the plurality of electromagnetic sensors 220 are installed in the second surface S2 that is the uneven surface. In other words, the second surface S2 may be configured with different planes, and positions and orientations of the plurality of electromagnetic sensors 220 may be different from each other.

Each of the plurality of electromagnetic sensors 220 may include a coil 222 through which an electromagnetic wave passes, and a processor 224. Here, the processor 224 may include an induced electromotive force detection circuit, and an analog-to-digital conversion circuit. The processing unit 230 may calculate the position and the orientation of each of the plurality of electromagnetic sensors 220 based on magnetic flux interlinked with the coil 222. The processing unit 230 may calculate the position and the orientation of a PSI based on the position and the orientation of each of the plurality of electromagnetic sensors 220.

In an example embodiment, magnetic flux interlinked with a coil 222 of each of the plurality of electromagnetic sensors 220 may have directivity corresponding to principal frequencies. Accordingly, magnetic fluxes corresponding to principal frequencies measured in each of the plurality of electromagnetic sensors 220 may be different in orientations and magnitudes. As a result, the processing unit 230 may obtain information about the position and the orientation of each of the plurality of electromagnetic sensors 220, to increase an accuracy of calculation of the position and the orientation of the PSI. This is more meaningful when the plurality of electromagnetic sensors 220 are installed on the second surface S2 that is the uneven surface of the PSI in different positions and orientations.

In an example embodiment, the plurality of electromagnetic sensors 220 may be spaced apart from each other at a set angular interval with respect to the PSI and may be installed on the second surface S2 that forms the uneven surface structure of the PSI, that is, in different planes. Thus, the processing unit 230 may calculate the position and the orientation of the PSI based on more accurate information about positions and orientations of the plurality of electromagnetic sensors 220 for the PSI.

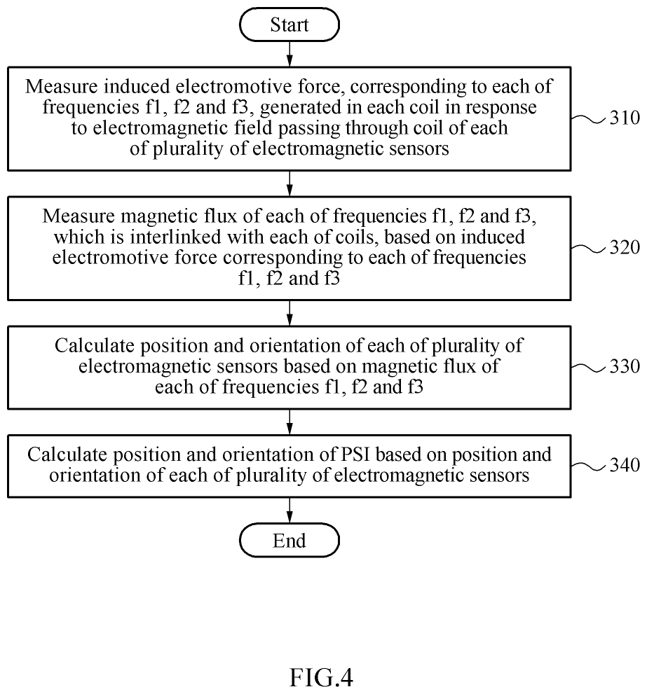

FIG. 4 is a flowchart schematically illustrating a method of registering coordinates of a PSI in a surgical navigation system for registering coordinates of a PSI according to an example embodiment.

Referring to FIG. 4, in operation 310, the method measures an induced electromotive force, corresponding to each of frequencies f1, f2 and f3, generated in each of coils, in response to an electromagnetic field passing through a coil of each of a plurality of electromagnetic sensors. In operation 320, the method measures magnetic flux of each of the frequencies f1, f2 and f3, which is interlinked with each of the coils, based on the induced electromotive force corresponding to each of the frequencies f1, f2 and f3. In operation 330, the method calculates the position and the orientation of each of the plurality of electromagnetic sensors based on the magnetic flux of each of the frequencies f1, f2 and f3. In operation 340, the method calculates the position and the orientation of the PSI based on the position and the orientation of each of the plurality of electromagnetic sensors. Here, information about the calculated position and the calculated orientation of the PSI may be used to register coordinates of the position of the PSI.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described example embodiments, or vice versa.

While this disclosure includes specific example embodiments, it will be apparent to one of ordinary skill in the art that various changes in form and details may be made in these example embodiments without departing from the spirit and scope of the claims and their equivalents. The example embodiments described herein are to be considered in a descriptive sense only, and not for purposes of limitation. Descriptions of features or aspects in each example embodiment are to be considered as being applicable to similar features or aspects in other example embodiments. Suitable results may be achieved if the described techniques are performed in a different order, and/or if components in a described system, architecture, device, or circuit are combined in a different manner and/or replaced or supplemented by other components or their equivalents.

1. A surgical navigation system for registering coordinates of a patient-specific instrument (PSI), the surgical navigation system comprising:
an electromagnetic wave generation unit configured to generate an electromagnetic wave;
an electromagnetic sensor installed in the PSI, the electromagnetic sensor comprising a coil through which the electromagnetic wave passes; and
a processing unit configured to calculate a position and an orientation of the electromagnetic sensor based on magnetic flux interlinked with the coil, and to calculate the position and the orientation of the PSI based on the position and the orientation of the electromagnetic sensor.

2. The surgical navigation system of claim 1, wherein the processing unit is configured to calculate the position and the orientation of the electromagnetic sensor based on frequencies orthogonal to each other in the magnetic flux interlinked with the coil.

3. The surgical navigation system of claim 2, wherein the processing unit is configured to calculate the magnetic flux based on an induced electromotive force generated in the coil in response to the electromagnetic wave passing through the coil.

4. The surgical navigation system of claim 1, wherein the processing unit is integrated with the electromagnetic sensor to be installed together in the PSI.

5. The surgical navigation system of claim 1, wherein the electromagnetic sensor further comprises:
an induced electromotive force detection unit configured to detect an induced electromotive force generated in the coil; and
an analog-to-digital conversion unit configured to convert the induced electromotive force from an analog form to a digital form

6. A surgical navigation system for registering coordinates of a patient-specific instrument (PSI), the surgical navigation system comprising:
an electromagnetic wave generation unit configured to generate an electromagnetic wave;
a plurality of electromagnetic sensors installed in the PSI, each of the plurality of electromagnetic sensors comprising a coil through which the electromagnetic wave passes, and coils being installed in different positions and orientations in different planes of the PSI; and
a processing unit configured to calculate a position and an orientation of each of the plurality of electromagnetic sensors based on magnetic flux interlinked with each of the coils, and to calculate the position and the orientation of the PSI based on the position and the orientation of each of the plurality of electromagnetic sensors.

7. The surgical navigation system of claim 6, wherein the processing unit is configured to calculate the position and the orientation of each of the plurality of electromagnetic sensors based on frequencies orthogonal to each other in the magnetic flux interlinked with each of the coils.

8. The surgical navigation system of claim 7, wherein the processing unit is configured to calculate the magnetic flux interlinked with each of the coils based on each induced electromotive force generated in each of the coils in response to the electromagnetic wave passing through each of the coils.

9. The surgical navigation system of claim 6, wherein the plurality of electromagnetic sensors are spaced apart from each other at a set angular interval with respect to the PSI and are installed in different planes.
